(19) United States
(12) Patent Application Publication
(10) Pub. No.: US 2025/0176972 A1
(43) Pub. Date: Jun. 5, 2025

(54) PRESSURE DELIVERY DEVICE FOR HEMORRHAGE CONTROL

(71) Applicant: REGENTS OF THE UNIVERSITY OF MICHIGAN, Ann Arbor, MI (US)

(72) Inventors: Kevin R. Ward, Superior Township, MI (US); Jeffrey Stephen Plott, Algonac, MI (US); Ethan A. Miles, Columbus, GA (US)

(21) Appl. No.: 18/962,838

(22) Filed: Nov. 27, 2024

Related U.S. Application Data
(60) Provisional application No. 63/604,591, filed on Nov. 30, 2023.

(51) Int. Cl. A61B 17/132; A61B 17/00; A61B 17/12

(52) U.S. Cl. CPC A61B 17/1325; A61B 2017/00477; A61B 2017/00862; A61B 2017/12004

(57) ABSTRACT

A portable compact hemorrhage occlusion includes a base and a pressure plate removably attached to the base. A strap carriage operably attached to the base and is linearly translatable relative to the base. A translation assembly is operably connected to the base and to the strap carriage. A strap is operably and directly connected to the strap carriage without being directly connected to the base. The translation assembly moves the strap carriage away from the base when the translation assembly is operated in a first direction and towards the base when the translation assembly is operated in a second direction.

### FIELD OF THE INVENTION

The disclosure generally relates to hemorrhage occlusion devices and more specifically to portable compact pressure delivery devices for junctional hemorrhage control that are capable of providing direct pressure and controlling hemorrhage at anatomical sites not amendable to treatment with traditional tourniquets.

### BACKGROUND OF THE INVENTION

Hemorrhage from vascular injuries in the extremities, such as the arms and legs, and/or from vascular injuries in the pelvis or abdomen can be difficult to treat by a single person. While the treatment of such injuries is challenging when they occur in civilian populations, treatment may be even more difficult in combat situations. Improvements in body armor have reduced mortality from combat injuries to the chest. However, the incidence of injuries to the extremities, pelvis, abdomen, axillary, and groin areas, and the associated mortality rates, remain high. Recent efforts have developed better hemorrhage control devices for treatment of these wounds.

Wounds to the axilla, groin, pelvis, and abdomen are complex and may involve several systems either alone or in combination, including major vascular structures, the bony pelvis, solid organs such as the liver and spleen, and even hollow organ injury to the bowel and bladder. Wounds directly involving isolated major vascular structures above the level of the femoral artery and vein such as the iliac artery and veins are most challenging to deal with followed by complex bony pelvic injuries from high velocity penetrating trauma resulting in complex arterial and lower pressure venous bleeding similar to those of blunt pelvic injuries experienced in a civilian trauma center.

Controlling hemorrhage by application of direct manual pressure may be particularly challenging in cases where the injured person is alone. In fact, most current tourniquet devices are designed to be applied “one-handed,” above the site of bleeding and not over the bleeding wound. However, it can be difficult and very painful to achieve a tourniquet pressure that stops blood flow with current tourniquet devices.

Even injuries involving isolated major vascular injury at or just above the inguinal ligament pose a tremendous field challenge in creating hemostasis. The femoral artery is usually palpable at the level of the inguinal ligament. Despite this, the ability to control bleeding by application of direct pressure by either the injured combatant or by others including fellow soldiers or medic aides will usually not suffice especially if rapid manual transport must take place. Controlling hemorrhage by application of direct manual pressure may be particularly challenging in cases where there is no large tissue defect allowing for packing and more pressure in closer proximity to the injured vessels. In fact, currently the only way to address this is by exploring the wound site, locating the artery and clamping it with hemostats. For deeper vascular injuries to the pelvis and abdomen, exploration is not an option until the time of surgery.

Even for hemorrhaging extremity wounds, application of packing materials into the wound followed by manual pressure or pressure applied by circumferential bandage wrapping can be difficult and ineffective. Application of traditional circumferential tourniquets directly over these wounds may not be effective and may result in complete cessation of blood flow distal to the wound creating limb ischemia.

### SUMMARY

In accordance with one exemplary aspect, a pressure delivery device for hemorrhage control includes a base and a pressure plate removably attached to the base. A strap carriage operably attached to the base and is linearly translatable relative to the base. A translation assembly is operably connected to the base and to the strap carriage. A strap is operably and directly connected to the strap carriage without being directly connected to the base. The translation assembly moves the strap carriage away from the base when the translation assembly is operated in a first direction and towards the base when the translation assembly is operated in a second direction.

In accordance with another exemplary aspect, a junctional tourniquet includes a base and a pressure plate removably attached to the base. A strap carriage is operably connected to the base and linearly translatable relative to the base. A translation assembly is operably attached to the base. A strap is operably and directly connected to the strap carriage without being directly connected to the base. A curved rigid back plate is operably connected to the strap. The translation assembly moves the strap carriage away from the base when the translation assembly is operated in a first direction and towards the base when the translation assembly is operated in a second direction.

In accordance with another exemplary aspect, a pressure delivery device for hemorrhage control includes a strap and a base operably connected to the strap. A pressure plate is removably attached to the base. A first peripheral ramp extends outward from a peripheral edge of the pressure plate. A strap carriage is linearly translatable relative to the base. A translation assembly is operably attached to the base. The translation assembly moves the strap carriage away from the base when the translation assembly is operated in a first direction and towards the base when the translation assembly is operated in a second direction.

In accordance with yet another exemplary aspect, a method of applying direct pressure to an object includes providing a pressure delivery device for hemorrhage control including a strap, a base, a strap carriage, and a translation assembly. A pressure plate is attached to a bottom of the base. The strap is wrapped around an object. The strap is operably and directly attached to the strap carriage, without directly attaching the strap to the base. The strap is fixed in position relative to the strap carriage. A rigid plate is disposed on a side of the object opposite the pressure delivery device for hemorrhage control. The strap is attached to the rigid plate. The strap carriage is moved away from the base, which causes the straps to tighten and produce pressure from a bottom of the pressure plate directly towards the object, and as the straps tighten, the straps and the rigid plate create a triangular-shape and the straps are spaced from the object proximate the pressure plate and proximate the sides of the rigid plate.

In further accordance with any one or more of the foregoing aspects, a pressure delivery device for hemorrhage control, a junctional tourniquet, or a method of applying direct pressure to an object, may further include any one or more of the following preferred forms.

In some preferred forms, the pressure plate comprises a deformable or moldable material.

In other preferred forms, a first peripheral ramp extends outward from a peripheral edge of the pressure plate and is angled relative to a central portion of the pressure plate.

In other preferred forms, the first peripheral ramp is angled away from a bottom of the pressure plate.

In other preferred forms, the first peripheral ramp is angled towards a bottom of the pressure plate.

In other preferred forms, the first peripheral ramp is angularly adjustable relative to the central portion.

In other preferred forms, a hinge operatively connects the central portion and the first peripheral ramp.

In other preferred forms, a deformable or moldable material operatively connects the central portion to the first peripheral ramp.

In other preferred forms, the first peripheral ramp may extend outward from a peripheral edge of the pressure plate up to about 4 inches, preferably between 1 and 4 inches, and more preferably about 2.5 inches.

In other preferred forms, the first peripheral ramp forms an angle of between 15 degrees and 60 degrees relative to a lateral or longitudinal axis of the pressure plate, preferably between 30 and 60 degrees, and more preferably about 40 degrees.

In yet other preferred forms, a rigid back plate is attached to the strap.

In yet other preferred forms, the rigid back plate is curved.

In yet other preferred forms, the curvature of the rigid back plate is defined by the two ends of the rigid back plate and a center of the rigid back plate lying on an arc having a radius of between 12 in and 40 in, preferably between 18 in and 26 in, and more preferably between 20 in and 24 in.

In yet other preferred forms, the back plate may be substantially flat, forming a substantially flat plane.

In yet other preferred forms, the translation assembly includes a screw assembly.

In yet other preferred forms, the screw assembly comprises nested screws.

In yet other preferred forms, the removable pressure plate includes two opposing rails.

In yet other preferred forms, the two opposing rails each include a wall and an overhang ledge, the base being captured between the walls of the opposing rails and between the overhang ledge and the pressure plate when the pressure plate is attached to the base.

In yet other preferred forms, the base includes a flexible arm that selectively secures the pressure plate to the base.

In yet other preferred forms, the removable pressure plate is removably attached to the base by press fit, hook and loop fasteners, a screw-on connection, a bayonet pin connection, or a rail and clip connection.

In yet other preferred forms, the strap carriage includes a first strap opening, and the strap is at least partially disposed in the first strap opening without being directly attached to the base.

### BRIEF DESCRIPTION OF THE DRAWINGS

FIG. 1 is a side perspective view of one embodiment of a pressure delivery device for hemorrhage control constructed in accordance with the teachings of the disclosure.

FIG. 2 is a side perspective view of the pressure delivery device for hemorrhage control of FIG. 1 with a pressure plate separated from a base.

FIG. 3 is a side view of the pressure delivery device for hemorrhage control of FIG. 1.

FIG. 4 is a top perspective view of the pressure delivery device for hemorrhage control of FIG. 1.

FIG. 5 is a posterior view of the pressure delivery device for hemorrhage control of FIG. 1, including a strap wrapped around an object, such as a limb, to provide directed pressure to the object, the pressure delivery device for hemorrhage control being in a deployed position that applies the directed pressure to the object.

FIG. 6 is a posterior view of the pressure delivery device for hemorrhage control of FIG. 1, including a strap wrapped around a second object, such as a junction of an abdomen and a leg, to provide directed pressure on the object, the pressure delivery device for hemorrhage control being in a deployed position that applies the directed pressure to the object.

FIG. 7 is a posterior view of the pressure delivery device for hemorrhage control of FIG. 1, including a strap wrapped around a third object, such as an abdomen, and including a rigid back plate to provide directed pressure to the object, the pressure delivery device for hemorrhage control being in a deployed position that applies the directed pressure to the object.

FIG. 8 is a posterior view of the pressure delivery device for hemorrhage control of FIG. 1, including a second pressure plate, and including a strap wrapped around the third object, also including a rigid back plate, the portable hemorrhage control device being in a retracted position, before being deployed, after being attached to the object.

FIG. 9 is a perspective view of the pressure delivery device for hemorrhage control of FIG. 8, without the strap.

FIG. 10 is a posterior view of the pressure delivery device for hemorrhage control of FIG. 8, in a deployed position, providing directed pressure to the object.

FIG. 11 is a top perspective view of a second embodiment of a rigid back plate.

FIG. 12 is another perspective view of the rigid back plate of FIG. 11.

FIG. 13 is a side view of the rigid back plate of FIG. 11.

FIG. 14 is a perspective view of another embodiment of a pressure delivery device for hemorrhage control constructed in accordance with the teachings of the disclosure.

FIG. 15 is a side view of the pressure delivery device for hemorrhage control of FIG. 14.

FIG. 16 is a front view of the pressure delivery device for hemorrhage control of FIG. 14.

### DETAILED DESCRIPTION

There is a need for a pressure delivery device that is capable of providing titrated direct downward pressure to various anatomical sites which optimize downward direct pressure and minimize creation circumferential pressure produced by traditional tourniquets.

The disclosed pressure delivery devices for hemorrhage control facilitate blood occlusion in patient bodies in emergency situations while enhancing comfort and intuitive use. The disclosed pressure delivery devices for hemorrhage control include a removable pressure plate (or plates) that may be selected based on a location and a size of a hemorrhage site.

A strap for the device may comprise a fabric, such as nylon or cotton. Other materials may also be used for the strap. The strap feeds through a pressure assembly which includes a screw assembly, a base, and a strap carriage.

The disclosed pressure delivery devices for hemorrhage control provide a portable, small-footprint device that can be used to selectively exert pressure on the body of an injured individual. The device is especially useful in emergency situations, and particularly to apply pressure to areas of the body where it is otherwise difficult to do so. A flexible support portion, such as a belt or strap may be circumferentially attached or secured to an area of the body of an individual in need of applied pressure (e.g. the pelvic area, abdominal area, the chest area, the axillary area, etc.). A pressure assembly is attached to the support portion. The attached pressure assembly is movable or positionable on the support portion i.e. the locations of the pressure assembly on the support portion are not fixed but are mobile, slidable, or otherwise adjustable. For example, the pressure assembly is slidable along a strap to more precisely target pressure to a hemorrhage location. In some embodiments, multiple pressure assemblies may be attached to the support portion to target multiple hemorrhage locations.

Upon activation of the pressure assembly, pressure is evenly distributed to the hemorrhage location immediately beneath a pressure plate. Counterexpansion away from the body's surface is prevented or significantly decreased by the nonexpandable nature of the support portion, i.e. the straps retain their dimensions and do not “stretch”.

The benefits of the pressure will vary from application to application. For example, in the case of uncontrolled bleeding from a non-compressible or difficult to compress location, blood vessels in the area are compressed, and bleeding from the compressed vessels is decreased or stopped. In one embodiment, the device is designed to be applied to peripheral areas such as the pelvic region (e.g. inguinal or groin area) to stop bleeding from, for example, femoral and external iliac blood vessels.

Another effect of the screw assemblies is that it also provides support (i.e. stability or rigidity) to the region of the body to which it is applied.

The deployment of the device is rapid and can be carried out by individuals with very little prior training. In fact, a wounded individual in need of such treatment may be able to deploy the device him or herself. The device may thus be used to provide support and/or to stop or lessen bleeding at a trauma site (e.g. on the battlefield, or at the scene of an accident) and during transport to a clinic or hospital where further medical treatment can be provided.

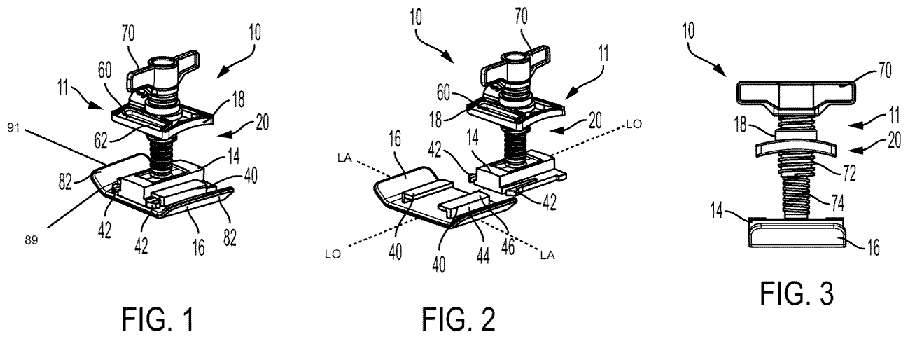

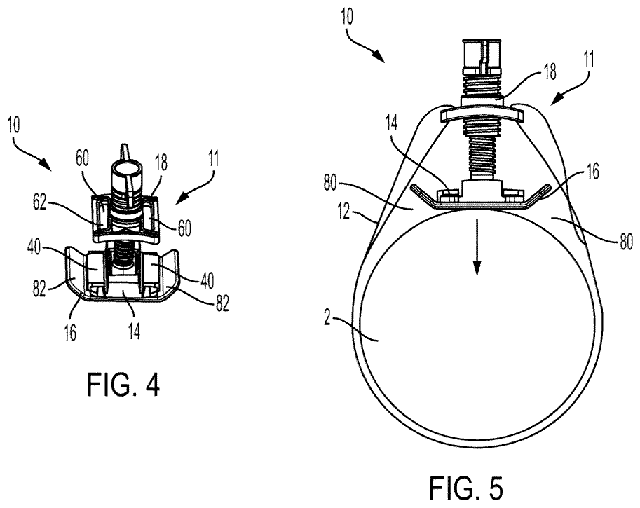

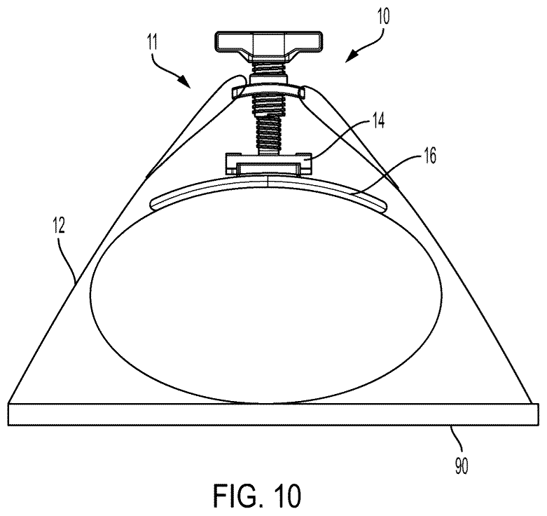

Turning now to the figures, specifically FIGS. 1-4 illustrate one embodiment an anti-hemorrhage system, which includes a pressure delivery device for hemorrhage control 10 (a strap for the device is not illustrated in FIGS. 1-4, but is illustrated in FIGS. 5-10). The pressure delivery device for hemorrhage control 10 includes a pressure assembly 11. The pressure assembly 11 comprises a base 14, a pressure plate 16 removably attached to the base 14, a strap carriage 18 linearly translatable relative to the base 14 and a translation assembly (in the illustrated embodiment, the translation assembly comprises a screw assembly 20) is attached to the base 14. While the translation assembly in the illustrated embodiment comprises a screw assembly 20, other translation assemblies may be used in other embodiments. For example, other embodiments may include hydraulic or pneumatic lifts, wedges, levers, or other assemblies that translate one element relative to another. The screw assembly 20 may have advantages over other types of translation assemblies. For example, the screw assembly 20 is relatively compact and provides a mechanical advantage that can produce increased pressure over other types of translation assemblies. The screw assembly 20 moves the strap carriage 18 away from the base 14 when the screw assembly 20 is operated in one direction and towards the base 14 when the screw assembly 20 is operated in an opposite direction.

The pressure plate 16 is removably attached to the base 14 so that pressure plates 16 having different shapes (e.g., FIG. 9) may be removed and replaced based on a needed fit or area to cover a hemorrhage location. The pressure plate 16 may include a connecting structure, such as two opposing rails 40 that guide the pressure plate 16 into position when installing the pressure plate 16 on the base 14. The base 14 includes one or more flexible arms 42 that selectively secures the pressure plate 16 to the base 14. The one or more flexible arms 42 form a clip that removably locks the pressure plate 16 to the base 14. In other embodiments, the pressure plate 16 may be removably attached to the base 14 with other types of connections, such as press fit, hook and loop fasteners, a screw-on connection, or a bayonet pin connection.

The two opposing rails 40 each include a wall 44 and an overhang ledge 46, the base 14 being captured between the walls 44 of the opposing rails 40 and between the overhang ledge 46 and the pressure plate 16 when the pressure plate 16 is attached to the base 14.

Turning now to FIGS. 5-7, in the deployed position, and when secured to one object, such as a limb 2 or an abdomen 3, the pressure plate 16 applies pressure towards the object, over a hemorrhage location, to occlude or reduce blood flow proximate the hemorrhage location. In some embodiments, wound packing (not shown) may be placed directly into the bleeding wound and located between the pressure plate 16 and the hemorrhage location. In such embodiments, the pressure delivery device 10 may be used to stop of slow hemorrhage by direct pressure, without needing to stop blood flow entirely to a limb including the hemorrhage location.

Returning now to FIGS. 1-4, the strap carriage 18 comprises at least one strap securing opening 60. In the illustrated embodiment, the strap carriage 18 comprises two strap securing openings 60, one for each strap 12 end. The strap securing openings 60 comprise slots 62 that are sized and shaped to receive at least a portion of the strap 12. More specifically, in the illustrated embodiment, each strap 12 end is threaded through its own slot 62.

The screw assembly 20 includes the handle 70 disposed at one end of the screw assembly 20. The screw assembly 20 includes a first screw 72 and a second screw 74 that is nested within the first screw 72. The first screw 72 has a larger diameter than the second screw 74. The first screw 72 may have a hollow central bore with female threads, the female threads cooperating with male threads on an outer surface of the second screw 74. In other embodiments, the male and female threads may be reversed.

Operating the screw assembly 20 (i.e., turning the handle 70 in a first direction) translates the strap carriage 18 away from the base 14 and causes the strap 12 to tighten and thus pressure to be applied by the pressure plate 16 in a downward direction (i.e., towards the object) in the figures.

The pressure plates 16 may include at least two individual pressure plates 16 having different shapes and/or different sizes. For example, a first pressure plate 16 (e.g., FIGS. 1-4) may have a square or rectangular shape and a second pressure plate 16 (e.g., FIG. 9) may have a triangular shape. Other regular or irregular shapes may also be included in the plurality of interchangeable pressure plates 16 to give an operator a variety of sizes and shapes to choose from to best suit the hemorrhage size and location. Any one of the pressure plates 16 may be removably attached, and thus interchanged with, the base 14. Moreover, in some embodiments, the pressure plate 16 may comprise a deformable or moldable material so that the pressure plate 16 shape may be modified to fit a treatment site or wound location. In some examples the pressure plate 16 may comprise a lower stiffness plastic such as, polyethylene, polypropylene, a thermoplastic elastomer, or combinations thereof. In other examples, a central portion of the pressure plate 16, where the pressure plate connects to the pressure delivery device, may comprise a more rigid plastic, while the sides of the pressure plate 16 may comprise more flexible materials. A bottom of the pressure plate 16, where the pressure plate 16 contacts a patient may, in some examples, comprise a softer material, such as a thermoplastic elastomer, to improve patient comfort and grip.

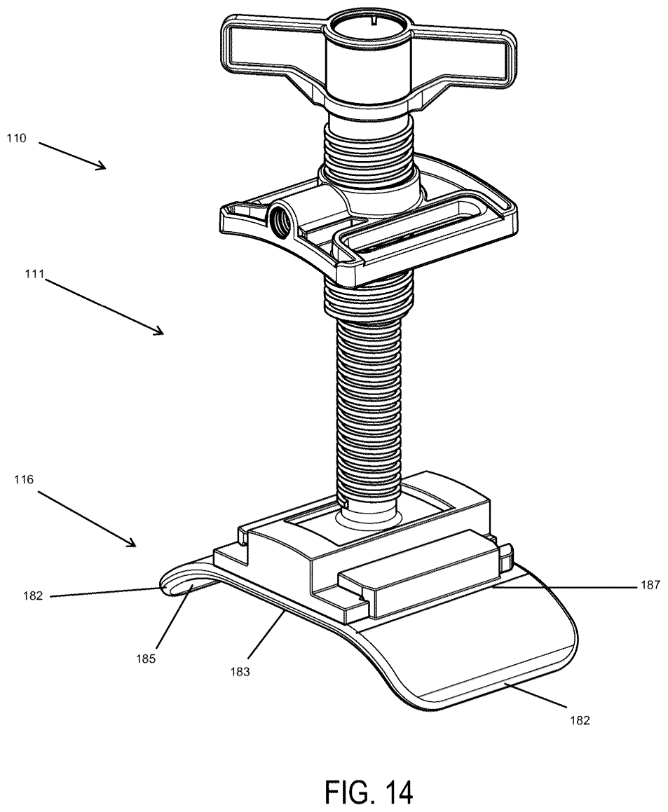

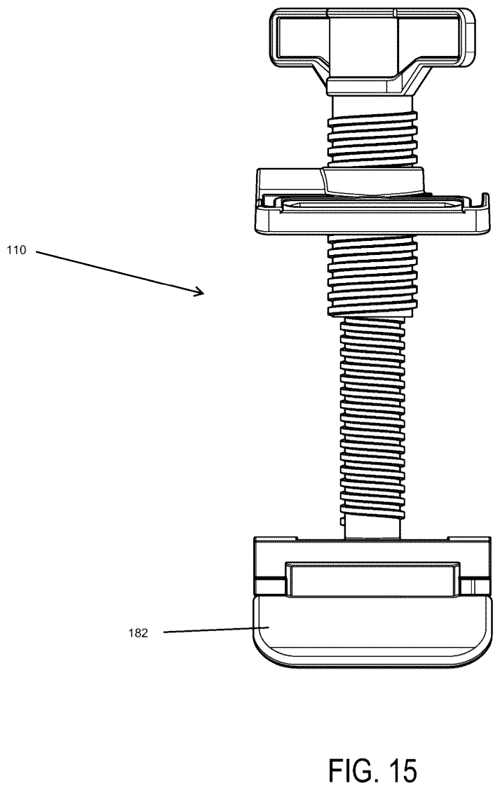

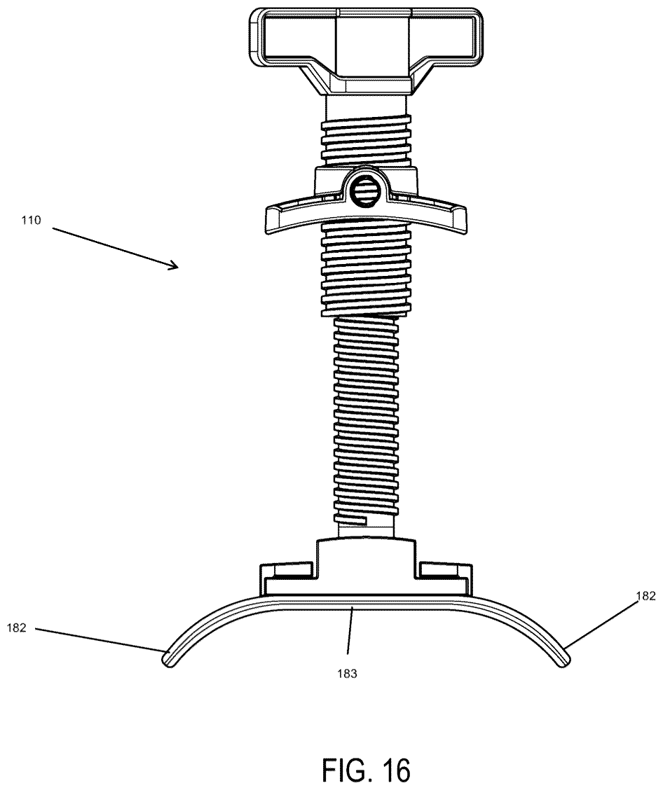

In one embodiment, the pressure plate 16 may include one or more inclined peripheral ramps 82 or “wings.” The inclined peripheral ramps 82 extend outward from a peripheral edge of the pressure plate 16. In some embodiment, the inclined peripheral ramps 82 may be substantially planar, while in other embodiments, the inclined peripheral ramps may be curved (either convexly or concavely). In the embodiment of FIGS. 1-4, the inclined peripheral ramps 82 are angled relative to a central portion 83 of the pressure plate 16. In some embodiments, the central portion 83 may have a substantially planar shape. In some embodiments, the inclined peripheral ramps 82 form an angle of between 15 and 60 degrees, preferably between 30 and 60 degrees, and more preferably about 40 degrees, with the pressure plate 16, relative to a lateral axis LA-LA or a longitudinal axis LO-LO of the pressure plate 16. In embodiments where the inclined peripheral ramps 82 are curved, the angle may be measured by forming a line between a proximal end 89 of the inclined peripheral ramp 82 and a distal end 91 of the inclined peripheral ramp 82. The inclined peripheral ramps 82 may angle away from a bottom 85 of the pressure plate 16 (as illustrated in FIGS. 1-4), or the inclined peripheral ramps 82 may angle towards the bottom 85 of the pressure plate 16 (as illustrated in FIGS. 14-16). The inclined peripheral ramps 82 may extend outward up to about 4 in, preferably between 1 in and 4 in, and more preferably about 2.5 in from the pressure plate 16. In some optional embodiments, each peripheral side of the pressure plate may include an inclined peripheral ramp 82. For example if the pressure plate 16 is substantially square or rectangular (as in FIGS. 1-4), a total of four inclined peripheral ramps 82 may extend from the pressure plate 16. The function of the inclined peripheral ramps 82 will be described further below.

The inclined peripheral ramps 82 may be angularly adjustable relative to the central portion 83. For example, in the embodiment illustrated in FIGS. 1-4, the inclined peripheral ramps 82 may be operatively connected to the central portion 83 by a hinge 87, which provides angular adjustability. In other embodiments, the inclined peripheral ramps 82 may be operatively connected to the central portion 83 by a moldable or deformable material.

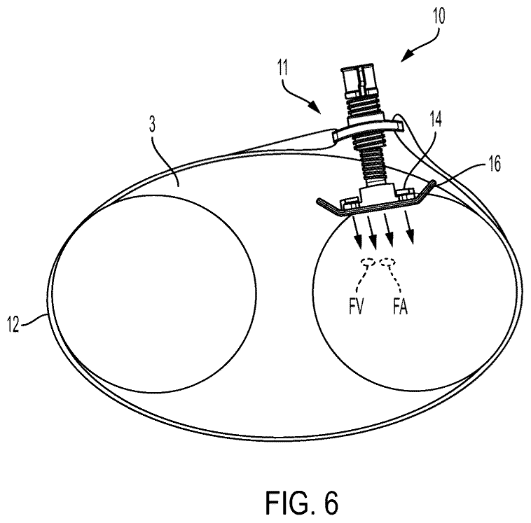

Turning now to FIGS. 5 and 6, in some cases it is desirable to direct pressure downward, into an object, for example into tissue of a limb 2 (FIG. 5), or into tissue near the junction of limbs 2 and the abdomen 3 (FIG. 6), to stop hemorrhages deep in the tissue, rather than to maximize circumferential pressure to stop all blood flow below a traditional the tourniquet location. For example, in FIG. 6, directed pressure (represented by arrows) is directed deep into the groin to reduce hemorrhage from the Femoral Artery (FA) and/or from the Femoral Vein (FV). The pressure delivery devices for hemorrhage control 10 disclosed herein maximize direct pressure by routing the strap 12 through the strap securing openings 60 without being directly connected to the base 14. In this way, when the strap carriage 18 begins to move away from the base 14, the ends of the strap 12 disposed in the strap securing openings 60 are pulled away from the object, creating space 80 between the strap 12 and the object 2 in the vicinity of the base 14, as illustrated in FIG. 5. The space 80 transfers some of the circumferential pressure from the strap 12 to direct pressure through the removable plate 16 and deep into the tissue. This directed pressure stops or reduces hemorrhage at the hemorrhage location without stopping blood flow to the rest of the limb 2. In this way, the hemorrhage in the limb 2 may be stopped or slowed, without tissue damage downstream of the tourniquet, which is a common result after a traditional tourniquet is placed upstream of the hemorrhage location. The deep directed pressure is also more effective in stopping deep tissue hemorrhages than traditional tourniquets, especially in junctional locations where a traditional circumferential torniquet is not effective. For example, the pressure delivery devices for hemorrhage control 10 disclosed herein may be especially useful in occluding hemorrhage in the femoral triangle and axillary areas, in the groin, in the axilla, in the neck, in the aorta, and/or in the abdomen and pelvis, or in any location where a traditional torniquet is difficult or impossible to locate.

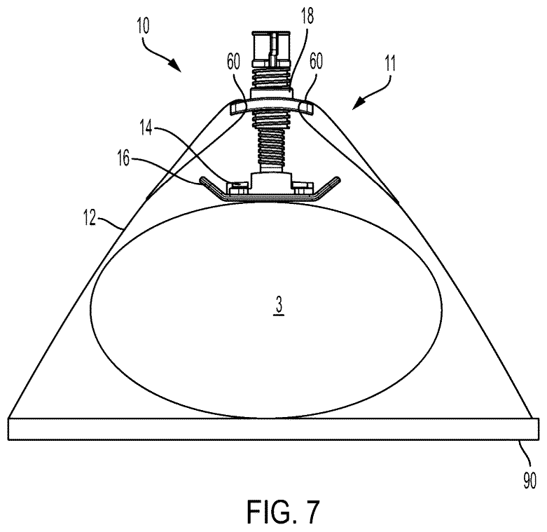

In configurations such as FIG. 7, the device is effective in partially or completely reducing blood flow at the level of the femoral artery and vein even when place on top of commonly available pelvic binders meaning that direct contact the skin is not necessary.

In configurations such as FIG. 7 for abdominal use the ability to fully or partially occlude the abdominal aorta is advantageous to reduce life threatening pelvic and/or lower extremity hemorrhage as well as post-partum hemorrhage. In addition, full or partial occlusion of the aorta may be helpful in increasing aortic diastolic pressure and thus coronary and cerebral perfusion pressure during cardiac arrest and during CPR as well as for patients with traumatic brain injury. Furthermore, this configuration can be utilized for deep pelvic traumatic hemorrhage or post-partum hemorrhage.

As the strap carriage 18 elevates above the pressure plate 16, the inclined peripheral ramps 82 stabilize and direct the pressure from the pressure plate 16 into the hemorrhage area. In other words, the inclined peripheral ramps 82 maintain proper location of the pressure plate 16 and prevent slipping or moving to keep the pressure directed in the appropriate location. While the primary purpose of the inclined peripheral ramps is to prevent slipping or to maintain directed pressure, in some optional embodiments, the inclined peripheral ramps 82 may also contact the strap 12 to assist in reducing circumferential pressure from the strap 12 and to further create space between the strap 12 and the limb 2, which further increases the directed pressure.

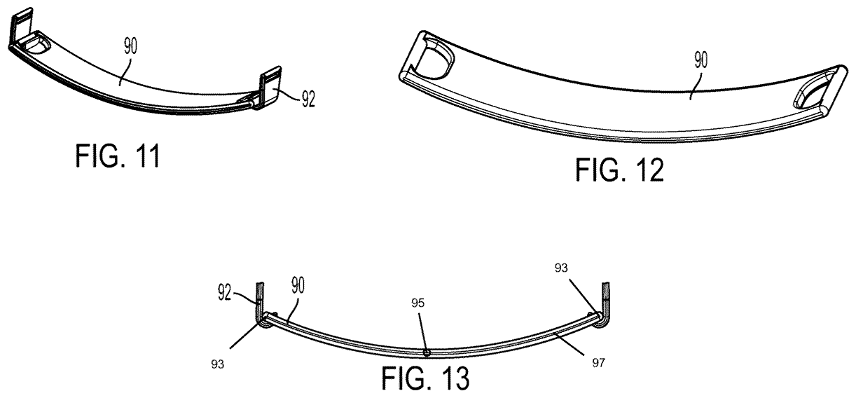

In another embodiment, the pressure delivery device for hemorrhage control 10 may include a rigid back plate 90, as illustrated in FIG. 7. The rigid back plate 90 may be especially useful in placements that are targeting aortic occlusion (sometimes referenced as aortic tourniquets). The rigid back plate 90 in FIG. 7 is generally a flat rigid plate. In some embodiments, the rigid back plate 90 may be curved, as illustrated in FIGS. 11-13. In some embodiments, the rigid back plate 90 may be between 10 and 30 inches wide and having an arc 97 defining a radius of curvature of between 12 in and 40 in, preferably between 18 in and 26 in, and more preferably between 20 in and 24 in, the radius of curvature being defined between two ends 93 and a center 95 of the rigid back plate 90.

The strap 12 is attached to each side of the rigid back plate 90. In some embodiments, the strap 12 may be attached directly to the rigid back plate 90 (as illustrated in FIGS. 7, 8, and 10), while in other embodiments, the strap 12 may be attached to the rigid back plate 90 with an adjustment mechanism, such as a J-hook 92 (as illustrated in FIGS. 11 and 13). As the strap 12 is pulled upwards by the strap carriage 18, the ends of the rigid back plate 90 keep the strap 12 spaced apart from the object 3, such as an abdomen. The tightened strap 12, the rigid back plate 90, and the pressure delivery device for hemorrhage control 10 form a triangular shape with the pressure delivery device for hemorrhage control 10 at the apex of the triangle. This triangular structure removes almost all circumferential pressure and transfers the circumferential pressure into direct pressure through the pressure plate 16.

More specifically, besides the space created between the strap 12 and the object 3 in the vicinity of the base plate 16, as described above, additional space is created proximate the sides of the rigid back plate 90. As a result, the pressure delivery device for hemorrhage control 10 takes the circumferential pressure of a traditional tourniquet and focuses the pressure almost entirely on two locations, one location being adjacent the pressure plate 16, and the other location being the rigid back plate 90 on the opposite side of the object 3 from the pressure plate 16. This causes a squeezing effect directly focused on the hemorrhage location, which more quickly occludes blood flow directly around the hemorrhage location. As a result, the pressure delivery device for hemorrhage control 10 may be used on parts of the body that are not receptive to traditional tourniquets, such as the abdomen or torso, because traditional tourniquets would cause collateral damage by reducing blood flow to essential organs.

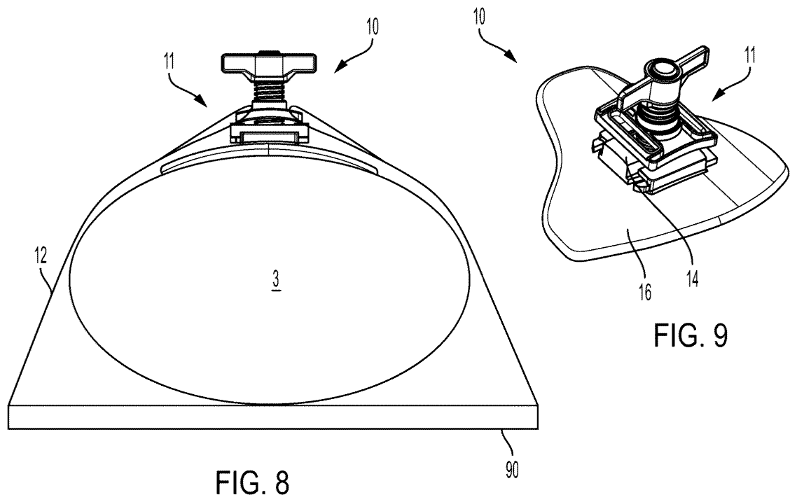

Turning now to FIGS. 8 and 9, another embodiment of a pressure delivery device for hemorrhage control 10 is illustrated that may be used on an object 3, such as an abdomen (although the pressure plate illustrated in FIG. 7 may also be used on the abdomen). The difference between the embodiment of FIGS. 8 and 9 and the embodiment illustrated in FIG. 7 is the shape of the pressure plate 16. Otherwise, the embodiment of FIGS. 8 and 9 operates essentially as the embodiment of FIG. 7. More specifically, the pressure plate 16 is curved in at least one plane, thereby creating a concave bottom surface that generally conforms with a convex shaped treatment site.

Turning now to FIGS. 14-16, an alternative embodiment of a pressure delivery device 110 is illustrated. The embodiment of FIGS. 14-16 differs from the embodiment of FIGS. 1-4 in the pressure plate 16. The remainder of the pressure assembly 11 is, at least in the illustrated example, identical to the embodiment of FIGS. 1-4 and will not be further discussed in the interest of brevity. The pressure plate 116 of FIGS. 14-16 includes two angled peripheral ramps 182. The angled peripheral ramps 182 are angled relative to a central portion 183. The angled peripheral ramps 182 are angled towards a bottom 185 of the pressure plate 116. The angled peripheral ramps 182 may be optionally angularly adjustable relative to the central portion 183. The angled peripheral ramps 182 may be operatively connected to the central portion 183 by a hinge 187 or other connection device that provides angular mobility. The embodiment of FIGS. 14-16 is especially well suited for application to wound locations having a relatively small radius of curvature, such as a lower arm or a lower leg, or over a joint, such as a knee or elbow. In such applications, the angled peripheral ramps 182 provide stability and resist movement off of the wound site.

General operation of the pressure delivery device for hemorrhage control 10 to provide direct pressure to stop a hemorrhage includes providing a pressure delivery device for hemorrhage control 10, as described above, including a strap 12, a base 14, a strap carriage 18, and a translation assembly 20. A pressure plate 16 is attached to a bottom of the base 14. The strap 12 is wrapped around an object 2,3, such as a limb or a torso. The strap 12 is operably and directly attached to the strap carriage 18, without directly attaching the strap 12 to the base 14. The strap 12 is fixed in position relative to the strap carriage 18. A rigid plate 90 is optionally disposed on a side of the object 2,3 opposite the pressure delivery device for hemorrhage control 10. The strap 12 is attached to the rigid plate 90. The strap carriage 18 is moved away from the base 14, which causes the strap 12 to tighten and produce pressure from a bottom of the pressure plate 16 directly towards the object 2,3, and as the strap 12 tightens, the strap 12 and the rigid plate 90 create a triangular-shape and the strap 12 is spaced from the object 2, 3, proximate the pressure plate 12 and proximate the sides of the rigid plate 90.

The pressure delivery devices for hemorrhage control 10 described above are particularly useful for occluding hemorrhages in body locations where conventional tourniquets are difficult to apply, for example, in the groin, abdomen, pelvis, and axilla. To occlude a hemorrhage location, the pressure delivery devices for hemorrhage control 10 described above are provided and attached to the patient body by wrapping the strap 12 around a portion of the patient body. The pressure assembly 11 is attached to the strap 12 by connecting the strap to the strap carriage 18. A pressure plate 16 is selected from the plurality of pressure plates 16 based on a location where pressure is needed. The pressure plate 16 is attached to the base 14. The screw assembly 20 is then operated to apply pressure to the patient body through the pressure plate 16.

In some embodiments, the screw assembly 20 is sized to provide approximately 1.75-3.5 inches of travel for the strap carriage 18, which is enough vertical travel to ensure that occlusion pressure is reached under all conditions.

In other embodiments, a writing surface may be provided for recording a time that the pressure delivery device for hemorrhage control is applied, which can be important information for a doctor or other medical person to know when evaluating treatment options.

The disclosed pressure delivery devices for hemorrhage control advantageously form a hemorrhage control system having a number of interchangeable plates (sizes and shapes) depending on the location of the hemorrhage. For example, smaller square/rectangular plates may be useful for hemorrhage locations in the groin or axilla, while larger triangular or square plates may be useful for the pelvis or abdomen. Moreover, the pressure delivery devices for hemorrhage control provide focused direct and deep pressure to a hemorrhage location, rather than circumferential pressure as in traditional tourniquets.

The disclosed pressure delivery devices for hemorrhage control advantageously may employ more than one base and plate at a time. For example, the disclosed pressure delivery devices for hemorrhage control may be configured to occlude a hemorrhage from a single penetrating injury to the groin or axilla and/or be configured to include plates to occlude hemorrhage from bilateral inguinal wounds plus a pelvic would (using two square plates for the inguinal founds and a large triangular plat for the pelvis).

The disclosed pressure delivery devices for hemorrhage control are easy to use, generate great mechanical advantage in tightening, and utilize wide bands or straps for less pain and more effective occluding pressures during application. The disclosed pressure delivery devices for hemorrhage control are also easily deployable and operable with one hand while operating solely on mechanical power generated by the user, so that a source of electrical power is not needed. Furthermore, due to the compact nature the disclosed pressure delivery devices for hemorrhage control are easily portable and generally light weight so that they may be deployed almost anywhere. The removable pressure plates facilitate optimizing the pressure location, which results occlusion pressure being applied more quickly, thereby minimizing blood loss.

While the present invention has been described with respect to a particular embodiment of the present invention, this is by way of illustration for purposes of disclosure rather than to confine the invention to any specific arrangement as there are various alterations, changes, deviations, eliminations, substitutions, omissions and departures which may be made in the particular embodiment shown and described without departing from the scope of the claims.

1. A pressure delivery device for hemorrhage control comprising:
a base;
a pressure plate removably attached to the base, the pressure plate including a central portion;
a strap carriage that is linearly translatable relative to the base;
a translation assembly that is operably connected to the base and to the strap carriage; and
a strap that is operably and directly connected to the strap carriage without being directly connected to the base,
wherein the translation assembly moves the strap carriage away from the base when the translation assembly is operated in a first direction and towards the base when the translation assembly is operated in a second direction.

2. The pressure delivery device for hemorrhage control of claim 1, wherein the pressure plate comprises a deformable or moldable material.

3. The pressure delivery device for hemorrhage control of claim 1, further comprising a first peripheral ramp extending outward from a peripheral edge of the pressure plate, the first peripheral ramp being angled relative to the central portion.

4. The pressure delivery device for hemorrhage control of claim 3, wherein the first peripheral ramp is angled away from a bottom of the pressure plate.

5. (canceled)

6. The pressure delivery device for hemorrhage control of claim 3, wherein the first peripheral ramp is angularly adjustable relative to the central portion.

7. The pressure delivery device for hemorrhage control of claim 6, further comprising a hinge operatively connected to the first peripheral ramp and the central portion, the hinge providing angular adjustability between the first peripheral ramp and the central portion.

8. (canceled)

9. The pressure delivery device of claim 3, wherein the first peripheral ramp extends outward from a peripheral edge of the pressure plate between 1 in and 4 in.

10. The pressure delivery device for hemorrhage control of claim 3, wherein the first peripheral ramp forms an angle of between 15 degrees and 60 degrees relative to a lateral axis or longitudinal axis of the pressure plate.

11. The pressure delivery device for hemorrhage control of claim 1, further comprising a rigid back plate attached to the strap.

12. The pressure delivery device for hemorrhage control of claim 11, wherein the rigid back plate is curved.

13. (canceled)

14. The pressure delivery device for hemorrhage control of claim 1, wherein the translation assembly comprises a screw assembly.

15. (canceled)

16. The pressure delivery device for hemorrhage control of claim 1, wherein the pressure plate includes two opposing rails.

17. The pressure delivery device for hemorrhage control of claim 16, wherein the two opposing rails each include a wall and an overhang ledge, the base being captured between the walls of the opposing rails and between the overhang ledge and the pressure plate when the pressure plate is attached to the base.

18. The pressure delivery device for hemorrhage control of claim 1, wherein the base includes a flexible arm that selectively secures the pressure plate to the base.

19. (canceled)

20. A junctional tourniquet comprising:
a base;
a pressure plate removably attached to the base, the pressure plate including a central portion;
a strap carriage operably connected to the base and linearly translatable relative to the base; and
a translation assembly that is operably attached to the base;
a strap operably and directly connected to the strap carriage without being directly connected to the base; and
a curved rigid pack plate operably connected to the strap;
wherein the translation assembly moves the strap carriage away from the base when the translation assembly is operated in a first direction and towards the base when the translation assembly is operated in a second direction.

21. The junctional tourniquet of claim 20, wherein the curvature of the rigid back plate is defined by the two ends of the rigid back plate and a center of the rigid back plate lying on an arc having a radius of between 4 in and 12 in, preferably between 7 in and 9 in.

22. The junctional torniquet of claim 20, further comprising a first peripheral ramp extending outward from a peripheral edge of the pressure plate, the first peripheral ramp being angled relative to the central portion.

23. (canceled)

24. The junctional torniquet of claim 20, wherein the strap carriage includes a first strap opening, and the strap is at least partially disposed in the first strap opening without being directly attached to the base.

25-29. (canceled)

30. A pressure delivery device for hemorrhage control comprising:
a strap;
a base operably connected to the strap;
a pressure plate removably attached to the base;
a first peripheral ramp extending outward from a peripheral edge of the pressure plate;
a strap carriage that is linearly translatable relative to the base; and
a translation assembly that is operably attached to the base,
wherein the translation assembly moves the strap carriage away from the base when the translation assembly is operated in a first direction and towards the base when the translation assembly is operated in a second direction.

31. (canceled)

32. A method of applying direct pressure to an object, the method comprising:
providing a pressure delivery device for hemorrhage control including a strap, a base, a removable pressure plate, a strap carriage, and a translation assembly;
wrapping the strap around an object;
operably and directly attaching the strap to the strap carriage, without directly attaching the strap to the base;
fixing the strap in position relative to the strap carriage;
disposing a rigid plate on a side of the object opposite the pressure delivery device for hemorrhage control;
attaching the strap to the rigid plate; and
moving the strap carriage away from the base, which causes the straps to tighten and produce pressure from a bottom of the pressure plate directly towards the object, and as the straps tighten, the straps and the rigid plate create a triangular-shape and the straps are spaced from the object proximate the pressure plate and proximate the sides of the rigid plate.

33. (canceled)
